(19) United States
(12) Patent Application Publication
(10) Pub. No.: US 2008/0161616 A1
(43) Pub. Date: Jul. 3, 2008

(54) Oxygenate to olefin processing with product water utilization

(76) Inventor: Lawrence W. Miller, Palatine, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/646,102

(22) Filed: Dec. 27, 2006

(51) Int. Cl. C07C 1/02; B01J 19/00

(52) U.S. Cl. 585/314; 422/187

(57) ABSTRACT

Processing schemes and arrangements for the production of olefins and, more particularly, for the production of light olefins from an oxygenate-containing feedstock are provided. Such processing schemes and arrangements offer improved energy utilization, additional light olefin products, and provide efficient uses for product water.

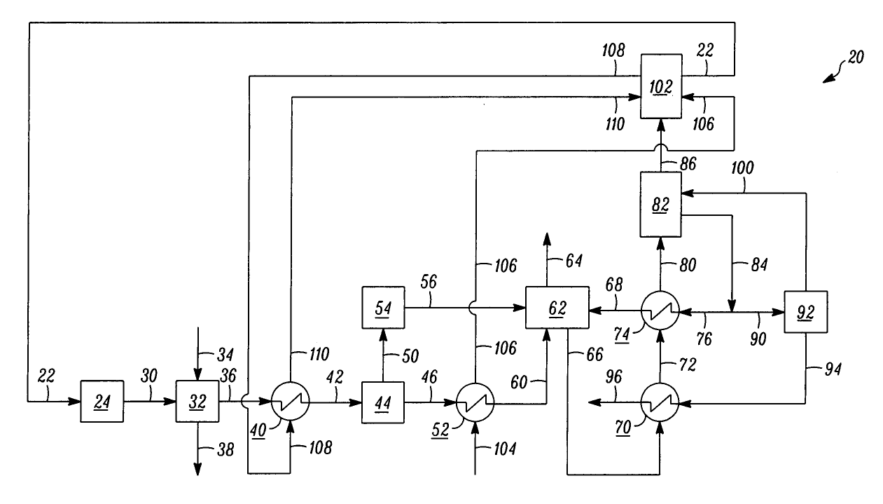

### BACKGROUND OF THE INVENTION

This invention relates generally to the conversion of oxygenates to olefins and, more particularly, to light olefins, via integrated processing.

A major portion of the worldwide petrochemical industry is involved with the production of light olefin materials and their subsequent use in the production of numerous important chemical products. Such production and use of light olefin materials may involve various well-known chemical reactions including, for example, polymerization, oligomerization, and alkylation reactions. Light olefins generally include ethylene, propylene and mixtures thereof. These light olefins are essential building blocks used in the modern petrochemical and chemical industries. A major source for light olefins in present day refining is the steam cracking of petroleum feeds. For various reasons including geographical, economic, political and diminished supply considerations, the art has long sought sources other than petroleum for the massive quantities of raw materials that are needed to supply the demand for these light olefin materials.

The search for alternative materials for light olefin production has led to the use of oxygenates such as alcohols and, more particularly, to the use of methanol, ethanol, and higher alcohols or their derivatives or other oxygenates such as dimethyl ether, diethyl ether, etc., for example. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

Such processing, wherein the oxygenate-containing feed is primarily methanol or a methanol-water combination (including crude methanol), typically results in the release of significant quantities of water upon the sought conversion of such feeds to light olefins. For example, such processing normally involves the release of about 2 mols of water per mol of ethylene formed and the release of about 3 mols of water per mol of propylene formed. The presence of such increased relative amounts of water can significantly increase the potential for hydrothermal damage to the oxygenate conversion catalyst. Moreover, the presence of such increased relative amounts of water significantly increases the volumetric flow rate of the reactor effluent, resulting in the need for larger sized vessels and associated processing and operating equipment.

U.S. Pat. No. 5,714,662 to Vora et al. discloses a process for the production of light olefins from a hydrocarbon gas stream by a combination of reforming, oxygenate production, and oxygenate conversion wherein a crude methanol stream (produced in the production of oxygenates and comprising methanol, light ends, and heavier alcohols) is passed directly to an oxygenate conversion zone for the production of light olefins.

In the past, conventional oxygenate to olefin processing schemes for handling product water separated from a hydrocarbon product stream included stripping the entire product water flow in a stripper operating in a severe stripping mode. As a consequence of such severe stripping mode of operating such processing consumed greater than desired quantities of energy.

While such processing has proven to be effective for olefin production, further improvements have been desired and sought. For example, there is an ongoing desire and need to minimize energy and utility consumptions. Further, there is an ongoing desire and need for processing schemes and arrangements that can more readily handle and manage product water associated with such processing. Still further, there is an ongoing desire and need for processing schemes and arrangements that produce or result in increased relative amounts of light olefins.

### SUMMARY OF THE INVENTION

A general object of the invention is to provide improved processing schemes and arrangements for the production of olefins, particularly light olefins.

A more specific objective of the invention is to overcome one or more of the problems described above.

The general object of the invention can be attained, at least in part, through specified processes for producing light olefins. In accordance with one embodiment, there is provided a process for producing light olefins from an oxygenate-containing feedstock that includes contacting the oxygenate-containing feedstock in an oxygenate conversion reaction system with an oxygenate conversion catalyst at effective conditions to form an oxygenate conversion effluent stream. The oxygenate conversion effluent stream includes a range of hydrocarbons including light olefins, water, and at least a quantity of effluent oxygenates. The effluent oxygenates include at least one of feedstock oxygenates, byproduct oxygenates, and intermediate oxygenates.

At least a portion of the oxygenate conversion effluent stream is contacted in a quench system with a quench water stream at effective conditions to remove heat from the oxygenate conversion effluent stream and to form a quench system stream. A product separation system separates at least a portion of the quench system stream at effective conditions to condense at least a quantity of water from the quench system stream. The separation in the product separation system also forms a product water stream comprising primarily water and forms a product stream comprising a range of hydrocarbons including light olefins and at least a quantity of effluent oxygenates.

The process also includes compressing at least a portion of the product stream in a compression system to form a compressed product stream. At least a portion of the compressed product stream is contacted in an oxygenate absorption system at effective conditions with a lean water stream and with at least a portion of the product water stream. The contacting in the oxygenate absorption system forms an absorber product stream comprising primarily a range of hydrocarbons including light olefins and forms a rich water stream comprising water and a quantity of effluent oxygenates.

At least a quantity of effluent oxygenates is stripped from at least a portion of the rich water stream in an oxygenate stripper system at effective conditions. Stripping in the oxygenate stripper forms an oxygenate recycle stream, comprising primarily a quantity of effluent oxygenates, and forms the lean water stream, comprising water and a reduced quantity of effluent oxygenates. At least a portion of the lean water stream is returned to the oxygenate absorption system.

The prior art generally fails to provide processing schemes and arrangements for the production of olefins and, more particularly, for the production of light olefins from an oxygenate-containing feed and which processing schemes and arrangements are as simple, effective and/or efficient as may be desired. More particularly, the prior art generally fails to provide such processing schemes and arrangements that address issues such as relating to water production, light olefin production, energy utilization and carbon efficiency for light olefin production as simply, effectively and/or efficiently as may be desired.

A process for producing light olefins, in accordance with another embodiment, involves producing light olefins from an oxygenate-containing feedstock. The process includes contacting the oxygenate containing feedstock in an oxygenate conversion reaction system with an oxygenate conversion catalyst at effective conditions to form an oxygenate conversion effluent stream comprising a range of hydrocarbons including light olefins, water, and a quantity of effluent oxygenates. The effluent oxygenates include feedstock oxygenates and intermediate oxygenates. The feedstock oxygenates include a quantity of methanol and the intermediate oxygenates include a quantity of dimethyl ether.

At least a portion of the oxygenate conversion effluent stream is contacted in a quench system with a quench water stream at effective conditions to remove heat from the oxygenate conversion effluent stream and to form a quench system stream. In a product separation system, at least a portion of the quench system stream is separated at effective conditions to condense at least a quantity of water from the quench system stream. The separating in the product separation system forms a product water stream comprising primarily water and forms a product stream comprising a range of hydrocarbons including light olefins and at least a quantity of effluent oxygenates.

The process further includes compressing at least a portion of the product stream in a compression system to form a compressed product stream. At least a portion of the compressed product stream is contacted in an oxygenate absorption system at effective conditions with a lean water stream and with at least a portion of the product water stream. The contacting in the oxygenate absorption system forms an absorber product stream comprising primarily a range of hydrocarbons including light olefins and forms a rich water stream comprising water and a quantity of effluent oxygenates.

At least a quantity of effluent oxygenates is stripped from at least a portion of the rich water stream in an oxygenate stripper system at effective conditions. The stripping in the oxygenate stripper forms an oxygenate recycle stream comprising primarily a quantity of effluent oxygenates, and forms the lean water stream comprising water and a reduced quantity of effluent oxygenates. The process includes returning at least a portion of the lean water stream to the oxygenate absorption system.

In a water stripper system, at least a quantity of effluent oxygenates is stripped from at least a portion of the lean water at effective conditions. The stripping in the water stripper forms a stripped water stream comprising primarily water and forms a stripper return stream comprising primarily a quantity of effluent oxygenates. The process includes returning at least a portion of the stripper return stream to the oxygenate stripper system.

There is also provided a system for producing light olefins from an oxygenate-containing feedstock. In accordance with one preferred embodiment, such a system includes an oxygenate conversion reaction system to contact the oxygenate-containing feedstock with an oxygenate conversion catalyst at effective conditions. In the oxygenate conversion reaction system, the contacting forms an oxygenate conversion effluent stream comprising a range of hydrocarbons including light olefins, water, and at least a quantity of effluent oxygenates. The effluent oxygenates include at least one of feedstock oxygenates, byproduct oxygenates, and intermediate oxygenates.

The system to produce light olefins from an oxygenate-containing feedstock includes a quench system to contact at least a portion of the oxygenate conversion effluent stream with a quench water stream at effective conditions to remove heat from the oxygenate conversion effluent stream and to form a quench system stream.

A product separation system is included to separate at least a portion of the quench system stream at effective conditions to condense at least a quantity of water from the quench system stream. The product separation system forms a product water stream comprising primarily water and forms a product stream comprising a range of hydrocarbons including light olefins and at least a quantity of effluent oxygenates.

At least a portion of the product stream is compressed in a compression system to form a compressed product stream. An oxygenate absorption system contacts at least a portion of the compressed product stream at effective conditions with a lean water stream and with at least a portion of the product water stream. The contacting in the oxygenate absorption system forms an absorber product stream comprising primarily a range of hydrocarbons including light olefins and to form a rich water stream comprising water and a quantity of effluent oxygenates.

An oxygenate stripper system strips at least a quantity of effluent oxygenates from at least a portion of the rich water stream at effective conditions. The stripping in the oxygenate stripper forms an oxygenate recycle stream comprising primarily a quantity of effluent oxygenates, and to form the lean water stream comprising water and a reduced quantity of effluent oxygenates. A return line returns at least a portion of the lean water stream to the oxygenate absorption system.

As used herein, references to “light olefins” are to be understood to generally refer to C₂ and C₃ olefins, i.e., ethylene and propylene.

In the subject context, the term “heavy olefins” generally refers to C₄-C₆ olefins.

“Oxygenates” are hydrocarbons that contain one or more oxygen atoms. Typical oxygenates include alcohols and ethers, for example.

“Carbon oxide” refers to carbon dioxide and/or carbon monoxide.

References to “Cₓ hydrocarbon” are to be understood to refer to hydrocarbon molecules having the number of carbon atoms represented by the subscript “x”. Similarly, the term “Cₓ-containing stream” refers to a stream that contains Cₓ-hydrocarbon. The term “Cₓ+hydrocarbons” refers to hydrocarbon molecules having the number of carbon atoms represented by the subscript “x” or greater. For example, “C₄+hydrocarbons” include C₄, C₅ and higher carbon number hydrocarbons. The term “Cₓ-hydrocarbons” refers to hydrocarbon molecules having the number of carbon atoms represented by the subscript “x” or less. For example, “C₄−hydrocarbons” include C₄, C₃ and lower carbon number hydrocarbons.

As used herein, references to “significant” with respect to a portion of dimethyl ether are to be understood to generally refer to at least about 75%, preferably at least about 90%, and more preferably at least about 95% of the identified element or elements.

As used herein, references to “primarily” with respect to hydrocarbons, oxygenates, and water alone or in combination are to be understood to generally refer to at least about 55%, preferably at least about 75%, and more preferably at least about 90% of the identified element or elements.

As used herein, references to “effluent oxygenates” with respect to an oxygenate conversion effluent stream and subsequent processing streams are the compounds that contain oxygen and carbon.

As used herein, references to “make up” with respect to feedstock, oxygenate, quench water and/or related streams can mean fresh, supply, and/or source of the identified stream and/or component.

Other objects and advantages will be apparent to those skilled in the art from the following detailed description taken in conjunction with the appended claims and drawings.

### BRIEF DESCRIPTION OF THE DRAWING

The FIG. is a simplified schematic diagram of an integrated system for the processing of an oxygenate-containing feedstock to olefins, particularly light olefins and including use of product water, in accordance with one embodiment.

Those skilled in the art and guided by the teachings herein provided will recognize and appreciate that the illustrated system or process flow diagram has been simplified by the elimination of various usual or customary pieces of process equipment including some heat exchangers, process control systems, pumps, fractionation systems, and the like. It may also be discerned that the process flow depicted in the figure may be modified in many aspects without departing from the basic overall concept of the invention.

### DETAILED DESCRIPTION

Oxygenate-containing feedstock can be converted to light olefins in a catalytic reaction and heavier hydrocarbons (e.g., C₄+hydrocarbons) formed during such processing can be subsequently further processed to increase the light olefins (e.g., C₂ and C₃ olefins) produced or resulting therefrom. In accordance with a preferred embodiment, a methanol-containing feedstock is converted to form dimethyl ether (DME) which in turn is reacted to form a product mixture including light olefins and heavy olefins, with at least a portion of the heavy olefins being subsequently converted to form additional light olefin products.

The FIG. schematically illustrates an integrated system, generally designated by the reference numeral 20, for processing of an oxygenate-containing feedstock to olefins, particularly light olefins, in accordance with one embodiment.

More particularly, an oxygenate-containing feedstock is introduced via a line 22 into an oxygenate conversion reaction system 24 wherein the oxygenate-containing feedstock contacts with an oxygenate conversion catalyst and, at reaction conditions effective to convert the oxygenate containing feedstock, produces an oxygenate conversion effluent stream via a line 30. The oxygenate conversion effluent stream may include a range of hydrocarbons including light olefins, water, and at least a quantity of effluent oxygenates including at least one of feedstock oxygenates, byproduct oxygenates, and intermediate oxygenates.

The range of hydrocarbons typically may desirably include light olefins as well as fuel gas hydrocarbons and C₄+hydrocarbons, including a quantity of heavy hydrocarbons. The water content of the effluent may include water produced from the oxygenate conversion reaction, water introduced with the makeup feedstock, and/or water associated with a recycle stream. The oxygenate conversion reaction system may include processing as is known in the art, such as, for example, utilizing a fluidized bed reactor.

The oxygenate-conversion feedstock may generally include compounds that include carbon and oxygen. Such classes of compounds may include alcohols, esters, ketones, aldehydes, carboxylic acids, other carbonyl containing species, other organic hydroxyl containing species, and their derivatives. Low molecular weight alcohols derived from natural gas supplies may provide a desirable feedstock. The oxygenate conversion feedstock may also be composed of a makeup feed and/or recycle streams. The fresh feeds and/or recycle streams may include water. As will be appreciated by those skilled in the art and guided by the teachings herein provided, it may be desirable to minimize the quantity of water fed to the reactor system to minimize vessel size and to provide longer catalyst life. In one embodiment the makeup is crude methanol with a methanol content of at least about 65 wt. %. Preferably the methanol content is at least about 80 wt. %, at least about 95 wt. %, or about 100 wt. %.

Effluent oxygenates may be present in the oxygenate conversion effluent and subsequent processing streams. Such oxygenates may include feedstock oxygenates, byproduct oxygenates, and intermediate oxygenates. The feedstock oxygenates in the effluent stream are any of the above identified types of compounds fed to the reactor but which compounds did not convert on initial or subsequent passes through the reaction system. Byproduct oxygenates are typically oxygen containing C₄+compounds such as alcohols or esters as well as any oxygen and carbon containing compounds that cannot not be readily converted to hydrocarbon compounds.

Intermediate oxygenates refer to those oxygenate materials that have begun but did not complete the step of conversion to form a hydrocarbon compound. Such intermediate oxygenates typically readily proceed to form a hydrocarbon product upon contact at reaction conditions with an oxygenate conversion catalyst. Such intermediate compounds may include alcohols, ethers, and esters. One such preferred intermediate compound that readily converts to form a hydrocarbon product is dimethyl ether. Dimethyl ether can also be a preferred feed according to certain embodiments.

Reaction conditions for the conversion of oxygenates such as dimethyl ether, methanol and combinations thereof, for example, to light olefins are known to those skilled in the art. Preferably, in accordance with particular embodiments, reaction conditions comprise a temperature between about 200° C. (about 392° F.) and about 575° C. (about 1,067° F.), more preferably between about 300° C. (about 512° F.) and about 550° C. (about 1,022° F.), and most preferably between about 400° C. (about 752° F.) and about 525° C. (about 977° F.). As will be appreciated by those skilled in the art and guided by the teachings herein provided, the reactions conditions are generally variable such as dependent on the desired products. The light olefins produced can have a ratio of ethylene to propylene of between about 0.5 and about 2.0 and preferably between about 0.75 and about 1.25. If a higher ratio of ethylene to propylene is desired, then the reaction temperature is higher than if a lower ratio of ethylene to propylene is desired. The preferred feed temperature range is between about 100° C. (about 212° F.) and about 300° C. (about 572° F.). More preferably the feed temperature range is between about 150° C. (about 302° F.) and about 250° C. (about 482° F.). In accordance with one preferred embodiment, the temperature is desirably maintained below about 210° C. (about 410° F.) to avoid or minimize thermal decomposition.

The reactor may operate in a pressure range of about 65 kPa gauge (about 9 psi gauge) to about 500 kPa gauge (about 73 psi gauge). A typical pressure range may include about 135 kPa gauge (about 20 psi gauge) to about 275 kPa gauge (about 40 psi gauge).

In accordance with certain preferred embodiments, it is particularly advantageous to employ oxygenate conversion reaction conditions including an oxygenate conversion reaction pressure of at least about 240 kPa absolute (about 35 psi absolute). In certain preferred embodiments, an oxygenate conversion reaction pressure in a range of about 240 kPa absolute (about 35 psi absolute) to about 580 kPa absolute (about 84 psi absolute) is preferred. Moreover, in certain preferred embodiments an oxygenate conversion reaction pressure of at least about 300 kPa absolute (about 44 psi absolute) and such as in a range of about 300 kPa absolute (about 44 psi absolute) to about 450 kPa absolute (about 65 psi absolute) may be preferred. Those skilled in the art and guided by the teachings herein provided will appreciate that through such operation at pressures higher than normally utilized in conventional oxygenate-to-olefin, particularly methanol-to-olefin (e.g., “MTO”) processing, significant reductions in reactor size (e.g., reductions in size of the oxygenate conversion reactor can be realized). For example, in view of the ratio of pressure between normal operation and higher pressure operation in accordance herewith, reductions in reactor size of at least about 20 percent or more, such as reductions in reactor size of about 33 percent or more can be realized through such higher pressure operation.

Those skilled in the art and guided by the teachings herein provided will appreciate certain reaction system equipment such as regenerator units, fluid beds, cyclones, filters, pumps, heat exchangers, catalyst recycle transport mechanisms and the like may be used to with this invention.

In practice, oxygenate conversions of at least about 90%, preferably of at least about 95% and, in at least certain preferred embodiments, conversions of 98% to 99% or more can be realized in such oxygenate-to-olefin conversion processing.

The oxygenate conversion effluent stream via the line 30 or at least a portion thereof is appropriately processed such as through a quench system 32 such as to form a resulting quench system stream represented by a line 36. Quench water in a line 34 contacts the effluent stream to desuperheat and partially condense the reactor effluent. Superheat includes typically the enthalpy of a stream above the requirements for vaporization. For example, water at atmospheric pressure boils at 100° C. (about 212° F.) to form steam or water vapor and is further heated to a temperature of 150° C. (about 302° F.) at the same pressure to become superheated. The process of removing heat may include lowering the temperature by removal of sensible heat or lowering enthalpy content by removal of latent heat. Additional functions of the quench may include pH adjustment by neutralizing byproduct organic acids such as acetic acid with a basic, alkaline, or caustic material, and/or removing catalyst fines entrained in the product stream. In one embodiment the quench water is circulated from a bottom of a distillation tower to an intermediate tray above a location of a feed inlet or nozzle that introduces the reactor effluent into the tower.

The quench water circulation may include the use of additional pumps and/or heat exchangers. Another stream sometimes referred to herein as a drag stream 38 may be drawn from the quench system to blowdown the system such as to remove catalyst fines, remove very heavy byproduct hydrocarbons, remove very heavy byproduct oxygenates, and/or remove neutralization products or salts. Very heavy hydrocarbons and very heavy oxygenates typically include C₇+ molecules.

Makeup water may be supplied to the quench system by using at least a portion of water condensed in a product separation system 44. The water condensation and operation of the product separation system 44 are more fully described below. Some of the water in the quench system may be vaporized and exit the system with the product stream in order to desuperheat the product stream. Make up water may also supply the excess water required to form the drag stream 38.

The quench system 32 typically comprises a vessel and such a vessel may have internal components including nozzles, plates, trays, random packing, structured packing, baffles, distributors, weirs, and the like as chosen by those skilled in the art. The operating conditions of the quench include a pressure range of about 50 kPa gauge (about 7 psi gauge) to about 100 kPa gauge (about 15 psi gauge) and a temperature range of about 80° C. (about 176° F.) to about 120° C. (about 248° F.). In one embodiment, the pressure may be about 83 kPa gauge (about 12 psi gauge) at the bottom of a quench tower and about 69 kPa gauge (about 10 psi gauge) at the top of the quench tower. The temperature may be about 109° C. (about 228° F.) at the bottom of the quench tower and about 107° C. (about 225° F.) at the top of the quench tower. Those skilled in the art and guided by the teachings herein provided will appreciate that increasing or decreasing reactor operating pressure will have a corresponding impact on the operating conditions of upstream and/or downstream process equipment.

At least a portion of the quench effluent is further processed to separate hydrocarbon compounds from water produced in the reactor. In a preferred embodiment and as illustrated in the FIG., at least a portion of the quench system stream represented by the line 36 is cooled and/or condensed in a heat transfer system 40. The cooling is by indirect contact wherein a first stream such as the quench system stream and a second stream such as a feedstock circulation stream do not physically mix. Such cooling or thermal energy transfer may be accomplished with the use of heat exchangers including double pipe, shell and tube, hair-pin, extended surface, plate and frame, spiral, single pass, multipass, and the like to produce thermal communication between fluids.

In this embodiment, the cooling also serves to heat at least a portion of a circulation stream taken from a feedstock flash system 102. The feedstock flash system 102 is more fully described below. Supply of the circulation stream is represented by a line 108 and return of the circulation stream is represented by a line 110. A cooled quench system stream, represented by a line 42, exits the heat transfer system 40 prior to the product separation system 44. Those skilled in the art and guided by the teachings herein provided will appreciate the benefits of heat integration of process streams to reduce utility consumption and favorably improve the overall process energy efficiency.

U.S. Pat. No. 6,459,009 to Miller et al. discloses a process for recovering heat and removing impurities that further details possible configurations for the heat transfer system 40 and related systems. Heat integration techniques may include the method referred to as pinch design. Pinch design involves typically the systematic analysis of temperature and enthalpy content of process streams and using such streams for heating and/or cooling requirements in other steps of the process to minimize a consumption of external utilities such as steam or cooling water.

Specifically in this present embodiment, cooling the quench system stream as represented by the line 36 may reduce a cooling duty on the product separator pump arounds.

The product separation system 44 separates water from the hydrocarbon product and oxygenates following the quench. In one embodiment, at least a portion of the cooled quench system stream represented by the line 42 is separated at conditions effective to condense at least a quantity of water from the cooled quench system stream introduced via the line 42 to form a product water stream represented by a line 46 comprising primarily of water and to form a product stream represented by a line 50 comprising a range of hydrocarbons including light olefins and at least a quantity of effluent oxygenates.

The product separation system 44 preferably includes pump arounds, side draw circuits, or circulation loops with pumps and heat exchangers for cooling the contents of the product separation system 44, such as, for example, to condense the product water. In one embodiment, the product separation system 44 is a distillation tower with at least one circulation loop and a plurality of separation stages. The circulation loop may be withdrawn from an intermediate separation stage, cooled and returned above the withdrawal separation stage. Those skilled in the art and guided by the teachings herein provided will appreciate that such circulation loops may be configured to be withdrawn and returned to locations from below the lowest separation stage to the above the highest separation stage and/or any intermediate separation stage in between and combinations thereof.

Additionally, those skilled in the art and guided by the teachings herein provided will appreciate that additional heat integration or pinch design may be employed to improve the energy efficiency of the process. In one embodiment, a circulation loop supplies heat to a propylene splitter reboiler in a heat exchanger, not illustrated. In another embodiment not illustrated, at least a portion of a propylene splitter system stream may be heated and at least a portion of the product water stream may be cooled by indirect contacting in the heat exchanger to vaporize at least a quantity of propylene. Other possible uses for low grade heat from the circulation loops may include warming a portion of a feedstock stream.

A portion of the product water, not illustrated, from the product separation system 44 may be supplied to the quench system 32. The product water supplied to the quench can be useful as a water wash to avoid caustic carryover to the downstream process equipment. Such supply may be on level control to regulate the makeup water flowing to the quench system.

In one embodiment not illustrated, a part of a circulation loop is supplied to an oxygenate absorption system 62 to assist in oxygenate recycle. The oxygenate absorption system 62 is more fully described below. A balance of the circulation loop flow may be returned to the product separation system 44 on flow control, not illustrated. Thus net water produced from the oxygenate conversion reaction may be sent to the oxygenate absorption system after makeup requirements to the quench and circulation requirements of the product separator are satisfied. The product water may contain low levels of oxygenates and/or hydrocarbons.

The product separation system 44 typically comprises a vessel and such a vessel may have internal components including nozzles, plates, trays, random packing, structured packing, distributors, baffles, weirs, and the like as selected by those skilled in the art. The operating conditions of the product separator may include a pressure range of about 25 kPa gauge (about 4 psi gauge) to about 75 kPa gauge (about 17 psi gauge) and a temperature range of about 35° C. (about 95° F.) to about 140° C. (about 284° F.). In one embodiment, the pressure may be about 55 kPa gauge (about 8 psi gauge) at the bottom of a product separator tower and about 41 kPa gauge (about 6 psi gauge) at the top the product separator tower. The temperature may be about 103° C. (about 217° F.) at the bottom of the product separator tower and about 43° C. (about 109° F.) at the top of the product separator tower.

Those skilled in the art and guided by the teachings herein provided will appreciate that the processing scheme illustrated in the FIG. decouples the product separator from a water stripper since product water is used in the oxygenate absorption system 62. In the past some designs have a water stripper taking product water from the product separator and returning a stream from the water stripper with effluent oxygenates to the product separator. The prior design may result in effluent oxygenates in the product water being stripped and returned to the product separator where they maybe condensed back in the product water by the cooling of the pump arounds. This internal reflux may result in a less efficient design and more contaminates in the product water than desired.

The illustrated configuration may reduce the likelihood of oxygenates being condensed in a product separator circulation loop and exiting with product water before being stripped in a water stripper and returned to the product separator. This illustrated configuration may reduce the likelihood of the product separator and the water stripper working against each other and offer incrementally improved product water quality.

The product water stream 46 may be used to heat or warm makeup or fresh oxygenate feedstock. As shown in the FIG. and according to a preferred embodiment, at least a portion of the product water stream via the line 46 is indirectly contacted in a heat transfer system 52 to cool at least a portion of the product water via the line 46 and heat the makeup feedstock via a line 104. The oxygenate makeup is represented by the line 104 and the warmed oxygenate makeup is represented by a line 106. The cooled product water stream, represented by a line 60 may be used in the oxygenate absorption system 62.

Those skilled in the art and guided by the teachings herein provided will appreciate that a cooled product water stream may increase a quantity of effluent oxygenates recovered from an absorber product stream. The cooling of the product water stream may lower the operating temperature in the oxygenate absorber thus improving the likelihood that oxygenates will be absorbed into a rich water stream. Effective cooling of the product water may include cooling to about ambient conditions, such as, for example, about 38° C. (about 100° F.).

The heat transfer system 52 may also cool at least a part of a product separator circulation loop, not illustrated. Suitable equipment for the heat transfer system 52 is discussed above with respect to the heat transfer system 40.

The process 20 further includes compressing at least a portion of the product stream via the line 50 in a compression system 54 to form a compressed product stream represented by a line 56. Those skilled in the art and guided by the teachings herein provided will appreciate that suitable compression equipment may include single or multistage compressors. Types of suitable compressors may include centrifugal, positive displacement, piston, diaphragm, screw, and the like. Suction, inter-stage, and discharge cooling and/or chilling along with corresponding liquid-vapor separation equipment may be included with such compression systems.

The compression system 54 is desirably capable of producing the pressures necessary for downstream processing such as used in conventional light olefin recovery units. Such recovery units may include a front end deethanizer wherein the first column of the recovery unit operates to remove ethane and lighter components from the balance of the column feed. The compressor discharge temperatures may be kept low to minimize compressor or equipment fouling. In one embodiment, the compression system is a centrifugal compressor with about three to about five stages. The final discharge pressure can be at least about 1,000 kPa gauge (about 145 psi gauge), preferably at least about 1,500 kPa gauge (about 217 psi gauge), and more preferably at least about 1,900 kPa gauge (about 275 psi gauge). In one embodiment, the discharge pressure is about 2,000 kPa gauge (about 290 psi gauge). The compressor discharge may be cooled to about ambient temperatures using conventional heat transfer methods.

As illustrated in the FIG. and according to a preferred embodiment, at least a portion of the compressed product stream via the line 56 is contacted in the oxygenate absorption system 62 at effective conditions to absorb at least a quantity of effluent oxygenates with a cooled lean water stream introduced via a line 68 and with at least a portion of the product water stream introduced via a line 60. The contacting in the oxygenate absorption system 62 forms an absorber product stream represented by a line 64 comprising primarily a range of hydrocarbons including light olefins and forms a rich water stream represented by a line 66 comprising water and a quantity of effluent oxygenates.

Use of the product water and the impurities therein in the oxygenate absorption system has minimal negative affect on the effectiveness of the system to recover oxygenates. A small amount of oxygenates such as methanol may help to more readily absorb certain oxygenates such as dimethyl ether into a liquid phase. In one embodiment, the lean water circulation is on flow control while the rich water circulation is on level control. Those skilled in the art and guided by the teachings herein provided will appreciate that the oxygenate absorption system may include one or more combinations of unit operation and/or mass transfer operation steps and/or equipment to achieve the desired results.

The oxygenate absorption system 62 serves to remove effluent oxygenates from the hydrocarbon product stream and allow such effluent oxygenates to be recycled to the reactor for improved feedstock utilization and economics. The oxygenate absorption system 62 is desirably capable of recovering at least about 75% of the entering oxygenates, preferably at least about 90% and more preferably at least about 95%. In one embodiment the oxygenate absorber recovers over 99% of the dimethyl ether that enters.

The absorber product stream via a line 64 may be sent to treating and processing such as may be necessary to recover select hydrocarbon fractions including light olefins. Such gas concentration units or gas plants are known in the art.

The oxygenate absorption system 62 may have operating conditions including a temperature range of about 30° C. (about 86° F.) to about 50° C. (about 122° F.) and a pressure range of about 1,500 kPa gauge (about 217 psi gauge) to about 2,000 kPa gauge (about 290 psi gauge). In one embodiment the oxygenate absorption system 62 temperature may be about 41° C. (about 106° F.) at the bottom of an absorber tower and about 40° C. (about 104° F.) at the top of the absorber tower. The pressure may be about 1,896 kPa gauge (about 275 psi gauge) at the bottom of the absorber tower and about 1,868 kPa gauge (about 270 psi gauge) at the top of the absorber tower.

A portion of a stripped water stream, not illustrated, may be returned to the oxygenate absorber system 62. The stripped water stream is more fully described below. Such a stripped water stream may be useful for providing a water wash at the top of the absorber to scrub remaining oxygenates from the absorber product stream.

In one embodiment as illustrated in the FIG., the rich water stream via the line 66 may indirectly contact a stripped water stream via a line 94 in a heat transfer system 70 to form a cooled stripped water stream represented by a line 96 and a heated rich water stream represented by a line 72. Suitable equipment for the heat transfer system 70 is discussed above with respect to the heat transfer system 40.

Those skilled in the art and guided by the teachings herein provided will appreciate that heating a rich water stream may increase a quantity of effluent oxygenates in an oxygenate stripper stream. The heating of the rich water stream may raise the temperature in the oxygenate stripper feed thus improving the likelihood that oxygenates will be stripped from the rich water and/or lowering the duty of an associated reboiler, such as, for example, reducing the energy and/or utility requirements of the reboiler.

The heated rich water stream 72 may further indirectly contact a lean water stream via a line 76 in a heat transfer system 74 to form a cooled lean water stream represented by the line 68 and a heated rich water stream represented by a line 80. Suitable equipment for the heat transfer system 74 is discussed above with respect to the heat transfer system 40. Those skilled in the art and guided by the teachings herein will appreciate that various schemes can be applied to exchange heat between the lean water stream, rich water stream, and/or stripped water stream to improve the efficiency of the design.

Those skilled in the art and guided by the teachings herein provided will appreciate that further heating a rich water stream may increase a quantity of effluent oxygenates in an oxygenate recycle stream. The heating of the rich water stream may raise the temperature in the oxygenate stripper feed thus improving the likelihood that oxygenates will be stripped from the rich water and/or lowering the duty of an associated reboiler, such as, for example, reducing the energy and/or utility requirements of the reboiler.

An oxygenate stripper system 82 at effective conditions to remove or strip effluent oxygenates from the circulation of rich water forms the lean water for use in the oxygenate absorption system 62. As illustrated in the FIG., the heated rich water stream via the line 80 is stripped in the oxygenate stripper system 82 to form an oxygenate stripper effluent represented by a line 84 comprising water and a reduced quantity of effluent oxygenates and to form an oxygenate recycle stream represented by a line 86 comprising primarily a quantity of effluent oxygenates. At least a portion of the oxygenate stripper effluent via the line 84 forms the lean water stream represented by a line 76 for return to the oxygenate absorption system 62 and another portion of the oxygenate stripper effluent may form a water stripper feed stream represented by a line 90. The flow of lean water can be on flow control per the requirements of the oxygenate absorber while the water stripper feed flow serves as level control for the oxygenate stripper system thus allowing net water to be sent to the water stripper.

Those skilled in the art and guided by the teachings herein provided will appreciate that the oxygenate stripper system 82 may include one or more combinations of unit operation and/or mass transfer operation steps and/or equipment to achieve the desired results. The oxygenate stripper system may have operating conditions including a temperature range of about 75° C. (about 167° F.) to about 175° C. (about 374° F.) and a pressure range of about 150 kPa gauge (about 22 psi gauge) to about 300 kPa gauge (about 44 psi gauge). In one embodiment the oxygenate stripper temperature may be about 136° C. (about 272° F.) at the bottom of a distillation tower and about 117° C. (about 243° F.) at the top of the distillation tower. The pressure may be about 234 kPa gauge (about 34 psi gauge) at the bottom of the distillation tower and about 221 kPa gauge (about 32 psi gauge) at the top of the distillation tower.

The process 20 may additionally include the step of stripping at least a quantity of effluent oxygenates from at least a portion of the lean water stream in a water stripper system at effective conditions to form a stripped water stream comprising primarily water and to form a stripper return stream comprising primarily a quantity of effluent oxygenates; and returning at least a portion of the stripper return stream to the oxygenate stripper system.

As illustrated in the FIG. and according to one embodiment, the stripper water feed stream via the line 90 is stripped in a water stripper system 92 to form a stripper return stream represented by a line 100 comprising primarily a quantity of effluent oxygenates and to form an stripper water stream represented by the line 94 comprising primarily water. The operating conditions of the water stripper system 92 desirably are more severe than the operating conditions of the oxygenate stripper system 82 such that an additional quantity of effluent oxygenates is removed from the feed. In one embodiment, a temperature at that bottom of the water stripper system 92 is above the bottom temperature of the oxygenate stripper system 82. Those skilled in the art and guided by the teachings herein will appreciate that the processing scheme illustrated in the FIG. can increase the efficiency of the oxygenate and water stripping systems by using the overhead from the water stripper to augment the stripping in the oxygenate stripper, whereas in previous designs the overhead merely increased the cooling duty of the product separation system.

Those skilled in the art and guided by the teachings herein provided will appreciate that suitable water stripper systems 92 may include conventional mass transfer operation and unit operation steps and/or equipment. The water stripper system 92 may have operating conditions including a temperature range of about 75° C. (about 167° F.) to about 150° C. (about 302° F.)and a pressure range of about 75 kPa gauge (about 11 psi gauge) to about 200 kPa gauge (about 29 psi gauge). In one embodiment the water stripper temperature may be about 128° C. (about 262° F.) at the bottom of a distillation tower and about 124° C. (about 255° F.) at the top of the distillation tower. The pressure may be about 152 kPa gauge (about 22 psi gauge) at the bottom of the distillation tower and about 131 kPa gauge (about 19 psi gauge) at the top of the distillation tower.

Additionally the process may include contacting at least a portion of the oxygenate recycle stream in a feedstock flash system with an oxygenate makeup to form the oxygenate-containing feedstock. According to one embodiment and as shown in the FIG., the feedstock flash system 102 contacts an oxygenate recycle stream via the line 86 with a warmed makeup feed stock via the line 106 to form the oxygenate-containing feedstock represented by the line 22 prior to contacting in the oxygenate conversion reaction system 24. Recycling effluent oxygenates to the reaction system improves the overall process yield and economics.

According to another embodiment not illustrated, the oxygenate make up stream prior to entering the feedstock flash system can be warmed in a first heat exchanger with a quench water stream before being warmed in a second heat exchanger with an overhead stream from the oxygenate stripper system.

A portion of the contents of the feedstock flash system may be circulated for heat integration as described above and designated by the line 108 for supply and the line 110 for return. If desired, the feedstock flash system may include an additional heat source and thereby function as a vaporizer.

Those skilled in the art and guided by the teachings herein provided will appreciate that suitable feedstock flash systems 102 may include conventional mass transfer and unit operation steps and/or equipment. Typical operating conditions for the feedstock flash system may include a pressure range of about 200 kPa gauge (about 29 psi gauge) to about 250 kPa gauge (about 36 psi gauge) and a temperature range of about 75° C. (about 167° F.) to about 140° C. (about 284° F.). In one embodiment the pressure may be about 221 kPa gauge (about 32 psi gauge) and the temperature may be about 101° C. (about 214° F.). The operating conditions of the feedstock flash system 102 may vary depending on the oxygenate conversion reactor design criteria, such as, for example, increased reactor pressures as described above may require a corresponding increase in feedstock flash system pressure. Typically, the feedstock flash system may operate at about 70 kPa gauge (about 10 psi gauge) above the pressure of the oxygenate reaction conversion system 24. The feedstock flash system may additionally include a return line, not illustrated, to the oxygenate stripper system 82 that may recycle an amount of methanol to the oxygenate stripper system 82 and remove solids from the feedstock flash system 102.

The invention thus provides processing schemes and arrangements for the production of olefins and, more particularly, for the production of light olefins from an oxygenate-containing feed and which processing schemes and arrangements are advantageously more efficient and provide a better use of product water than heretofore been generally available. An improved oxygenate recycle increases utilization of oxygenate-containing feeds and improved heat integration reduces energy consumption.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A process for producing light olefins from an oxygenate-containing feedstock, the process comprising:
contacting an oxygenate-containing feedstock in an oxygenate conversion reaction system with an oxygenate conversion catalyst at effective conditions to form an oxygenate conversion effluent stream comprising a range of hydrocarbons including light olefins, water, and at least a quantity of effluent oxygenates including at least one of feedstock oxygenates, byproduct oxygenates, and intermediate oxygenates,
contacting at least a portion of the oxygenate conversion effluent stream in a quench system with a quench water stream at effective conditions to remove heat from the oxygenate conversion effluent stream and to form a quench system stream;
separating at least a portion of the quench system stream in a product separation system at effective conditions to condense at least a quantity of water from the quench system stream to form a product water stream comprising primarily water and to form a product stream comprising a range of hydrocarbons including light olefins and at least a quantity of effluent oxygenates;
compressing at least a portion of the product stream in a compression system to form a compressed product stream;
contacting at least a portion of the compressed product stream in an oxygenate absorption system at effective conditions with a lean water stream and with at least a portion of the product water stream to form an absorber product stream comprising primarily a range of hydrocarbons including light olefins and to form a rich water stream comprising water and a quantity of effluent oxygenates;
stripping at least a quantity of effluent oxygenates from at least a portion of the rich water stream in an oxygenate stripper system at effective conditions to form an oxygenate recycle stream, comprising a quantity of effluent oxygenates, and to form the lean water stream, comprising water and a reduced quantity of effluent oxygenates; and
returning at least a portion of the lean water stream to the oxygenate absorption system.

2. The process of claim 1 additionally comprising:
stripping at least a quantity of effluent oxygenates from at least a portion of the lean water stream in a water stripper system at effective conditions to form a stripped water stream comprising primarily water and to form a stripper return stream comprising a quantity of effluent oxygenates; and
returning at least a portion of the stripper return stream to the oxygenate stripper system.

3. The process of claim 1 additionally comprising contacting at least a portion of the oxygenate recycle stream in a feed stock flash system with an oxygenate makeup to form the oxygenate-containing feedstock.

4. The process of claim 3 additionally comprising:
circulating at least a portion of the contents of the feedstock flash system to form a feedstock circulation stream; and
heating at least a portion of the feedstock circulation stream and cooling at least a portion of the quench system stream by indirect contacting in a first heat transfer system.

5. The process of claim 3 additionally comprising heating at least a portion of the oxygenate make up stream and cooling at least a portion of the product water stream by indirect contacting in a second heat transfer system to increase a quantity of effluent oxygenates removed from the absorber product stream.

6. The process of claim 2 additionally comprising heating at least a portion of the rich water stream and cooling at least a portion of the stripped water stream by indirect contacting in a third heat transfer system to increase a quantity of effluent oxygenates in the oxygenate recycle stream.

7. The process of claim 1 additionally comprising heating at least a portion of the rich water stream and cooling at least a portion of the lean water stream by indirect contacting in a fourth heat transfer system to increase a quantity of effluent oxygenates in the oxygenate recycle system stream.

8. The process of claim 1 additionally comprising returning a portion of the product water stream to the quench system.

9. The process of claim 1 wherein the product separation system comprises a column with a plurality of separation stages and a bottom portion, the product water stream is withdrawn from at least one location selected from the group consisting of a separation stage, the bottom portion, and combinations thereof.

10. The process of claim 1 additionally comprising heating at least a portion of a propylene splitter system stream and cooling at least a portion of the product water by indirect contacting in a heat exchanger to vaporize at least a quantity of propylene.

11. The process of claim 1 additionally comprising returning at least a portion of the stripped water stream to the oxygenate absorption system.

12. The process of claim 1 wherein the feedstock oxygenates comprise a quantity of methanol.

13. The process of claim 1 wherein the effluent oxygenates include intermediate oxygenates and the intermediate oxygenates comprise a quantity of dimethyl ether.

14. The process of claim 13 wherein the contacting in the oxygenate absorption system is effective to absorb a significant quantity of dimethyl ether from the contacted portion of the compressed product stream and to form the rich water stream.

15. A process for producing light olefins from an oxygenate-containing feedstock, the process comprising:
contacting an oxygenate-containing feedstock in an oxygenate conversion reaction system with an oxygenate conversion catalyst at effective conditions to form an oxygenate conversion effluent stream comprising a range of hydrocarbons including light olefins, water, and a quantity of effluent oxygenates including feedstock oxygenates and intermediate oxygenates, the feedstock oxygenates comprising a quantity of methanol and the intermediate oxygenates comprising a quantity of dimethyl ether;
contacting at least a portion of the oxygenate conversion effluent stream in a quench system with a quench water stream at effective conditions to remove heat from the oxygenate conversion effluent stream and to form a quench system stream;
separating at least a portion of the quench system stream in a product separation system at effective conditions to condense at least a quantity of water from the quench system stream to form a product water stream comprising primarily water and to form a product stream comprising a range of hydrocarbons including light olefins and at least a quantity of effluent oxygenates;
compressing at least a portion of the product stream in a compression system to form a compressed product stream;
contacting at least a portion of the compressed product stream in an oxygenate absorption system at effective conditions with a lean water stream and with at least a portion of the product water stream to form an absorber product stream comprising primarily a range of hydrocarbons including light olefins and to form a rich water stream comprising water and a quantity of effluent oxygenates;
stripping at least a quantity of effluent oxygenates from at least a portion of the rich water stream in an oxygenate stripper system at effective conditions to form an oxygenate recycle stream, comprising a quantity of effluent oxygenates, and to form the lean water stream, comprising water and a reduced quantity of effluent oxygenates;
returning at least a portion of the lean water stream to the oxygenate absorption system;
stripping at least a quantity of effluent oxygenates from at least a portion of the lean water stream in a water stripper system at effective conditions to form a stripped water stream comprising primarily water and to form a stripper return stream comprising a quantity of effluent oxygenates; and
returning at least a portion of the stripper return stream to the oxygenate stripper system.

16. The process of claim 15 additionally comprising:
contacting at least a portion of the oxygenate recycle stream in a feedstock flash system with an oxygenate makeup to form the oxygenate-containing feedstock;
heating at least a portion of the oxygenate makeup stream and cooling at least a portion of the quench system stream by indirect contacting in a first heat transfer system;
circulating at least a portion of the contents of the feedstock flash system to from a feedstock circulation stream; and
heating at least a portion of the feedstock circulation stream and cooling at least a portion of the product water stream by indirect contacting in a second heat transfer system to increase a quantity of effluent oxygenates removed from the absorber product stream.

17. The process of claim 15 additionally comprising:
heating at least a portion of the rich water stream and cooling at least a portion of the lean water stream by indirect contacting in a third heat transfer system to increase a quantity of effluent oxygenates in the oxygenate recycle stream; and
heating at least a portion of the rich water stream and cooling at least a portion of the stripped water stream by indirect contacting in a fourth heat transfer system to increase a quantity of effluent oxygenates in the oxygenate recycle stream.

18. The process of claim 15 additionally comprising:
returning a portion of the product water stream to the quench system; and
returning at least a portion of the stripped water stream to the oxygenate absorption system.

19. The process of claim 15 wherein the product separation system comprises a column with a plurality of separation stages and a bottom portion, the product water stream is withdrawn from at least one location selected from the group consisting of a separation stage, the bottom portion, and combinations thereof.

20. A system for producing light olefins from an oxygenate-containing feedstock, the process comprising:
an oxygenate conversion reaction system to contact an oxygenate-containing feedstock with an oxygenate conversion catalyst at effective conditions to form an oxygenate conversion effluent stream comprising a range of hydrocarbons including light olefins, water, and at least a quantity of effluent oxygenates including at least one of feedstock oxygenates, byproduct oxygenates, and intermediate oxygenates, a quench system to contact at least a portion of the oxygenate conversion effluent stream with a quench water stream at effective conditions to remove heat from the oxygenate conversion effluent stream and to form a quench system stream;
a product separation system to separate at least a portion of the quench system stream at effective conditions to condense at least a quantity of water from the quench system stream to form a product water stream comprising primarily water and to form a product stream comprising a range of hydrocarbons including light olefins and at least a quantity of effluent oxygenates;
a compression system to compress at least a portion of the product stream to form a compressed product stream;
an oxygenate absorption system to contact at least a portion of the compressed product stream at effective conditions with a lean water stream and with at least a portion of the product water stream to form an absorber product stream comprising primarily a range of hydrocarbons including light olefins and to form a rich water stream comprising water and a quantity of effluent oxygenates;
an oxygenate stripper system to strip at least a quantity of effluent oxygenates from at least a portion of the rich water stream at effective conditions to form an oxygenate recycle stream, comprising a quantity of effluent oxygenates, and to form the lean water stream, comprising water and a reduced quantity of effluent oxygenates; and
a return line to return at least a portion of the lean water stream to the oxygenate absorption system.
